(19) United States
(12) Patent Application Publication
(10) Pub. No.: US 2018/0171043 A1
(43) Pub. Date: Jun. 21, 2018

(54) XANTHENE-BASED POLYMERIC COMPOUNDS, A RESIN COMPOSITION FOR COLOR FILTER COMPRISING SAME AND A COLOR FILTER USING SAME

(71) Applicant: (KISCO), Incheon (KR)

(72) Inventors: Soon Hyun PARK, Gyeonggi-do (KR); Jeong Gi KIM, Gyeonggi-do (KR); Youn A LEE, Seoul (KR); Do Kyung LEE, Incheon (KR); Min Jung LEE, Seoul (KR); Dongil CHOI, Seoul (KR); Jung Rok KIM, Gyeonggi-do (KR)

(21) Appl. No.: 15/738,743

(22) PCT Filed: May 3, 2016
(86) PCT No.: PCT/KR2016/004646

§ 371 Date: Dec. 21, 2017

(30) Foreign Application Priority Data
Jun. 23, 2015 (KR) 10-2015-0089234

(51) Int. Cl. C08F 20/36; C08F 20/38; C07D 311/82; C08L 25/18; C08F 26/06; C08K 5/435; G02B 5/28

(52) U.S. Cl. CPC C08F 20/36; C08F 20/38; C07D 311/82; C09B 57/14; C08F 26/06; C08K 5/435; G02B 5/287; C08L 25/18

(57) ABSTRACT

The present invention relates to a polymeric compound used to a colored resin composition for a color filter, which contains a structure represented by Chemical Formula 1 or Chemical Formula 2 and has excellent heat resistance and light resistance and improved chemical resistance. Therefore, the compound can be applied to various materials, particularly, a display material such as a resin composition for a color filter.

### TECHNICAL FIELD

The present invention relates to a xanthene-based polymeric compound, a resin composition for a color filter comprising same and a color filter using same. More specifically, the present invention relates to a xanthene-based polymeric compound which is excellent in all of heat resistance, light resistance and chemical resistance.

### BACKGROUND ART

A liquid crystal display device displays images using optical and electrical properties of a liquid crystal material. The liquid crystal display device is advantageous over CRTs, plasma display panels and the like, in terms of low weight, low power consumption and low driving voltage. The liquid crystal display device includes a liquid crystal layer disposed between glass substrates. Light produced by a light source passes through the liquid crystal layer and the liquid crystal layer controls light transmittance. After passing through the liquid crystal layer, the light passes through a color filter layer. A full-color display is realized through additive color mixing of light that has passed through the color filter layer.

In general, in order to manufacture a color filter, after preparing a photoresist-type resin composition of RGB colors, an RGB pattern is formed by using a photolithography technique which repeatedly proceeds several processes such as coating on a glass substrate by RGB colors, photocuring, postbaking, developing and the like.

At this time, heat resistance and chemical resistance of a coloring agent are very important because processes such as exposing, postbaking, developing and the like are repeatedly proceeded for pattern formation.

Recently, as the demand for a color filter having high brightness, high color reproduction and high contrast ratio increases, development for a colored resin composition for a color filter having excellent heat resistance, light resistance and chemical resistance is urgently needed.

### DISCLOSURE

### Technical Problem

In order to solve the above-mentioned problems, the present invention provides a colored resin composition for a color filter which comprises a xanthene-based polymeric compound with excellent heat resistance, light resistance and chemical resistance.

### Technical Solution

In order to solve the above-mentioned technical problem, in an aspect of the present invention, a resin composition for a color filter may comprise a compound selected from xanthene-based polymeric compounds which contain a repeating unit of the following Chemical Formula 1 or Chemical Formula 2.

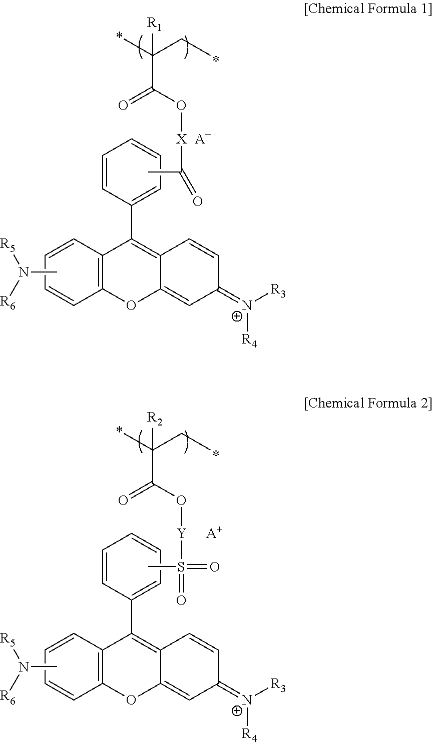

Specific substituents and structural characteristics of Chemical Formula 1 or Chemical Formula 2 will be described later.

Further, the polymeric compound of the present invention containing the xanthene-based structure represented by Chemical Formula 1 or Chemical Formula 2 may further contain a structure selected from structures of the following Chemical Formula 3 to Chemical Formula 8.

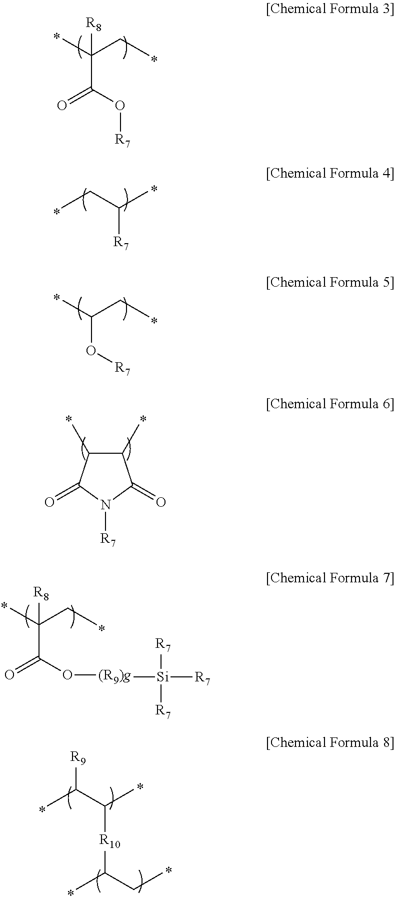

Further, in another aspect of the present, a resin composition for a color filter may comprise a coloring agent comprising a polymeric compound, a binder resin, a reactive unsaturated compound, a polymerization initiator, an organic solvent and additives, wherein the polymeric compound is the polymeric compound according to the present invention.

### Advantageous Effects

The polymeric compound according to the present invention is a polymeric compound synthesized by polymerizing a xanthene-based compound with excellent heat resistance and light resistance as a coloring agent, and it is characterized by having excellent heat resistance, light resistance and chemical resistance by improving poor chemical resistance of the conventional xanthene-based compound. Therefore, the compound can be useful for a coloring agent of a color filter resin composition.

### BEST MODE CARRYING OUT THE INVENTION

Hereinafter, the present invention will be described in detail. In one aspect, the present invention relates to a polymeric compound suitable for a resin composition for a color filter, and the compound is characterized by having excellent heat resistance, light resistance, chemical resistance and the like.

The polymeric compound according to the present invention is characterized by containing a xanthene-based compound structure represented by the following Chemical Formula 1 or Chemical Formula 2.

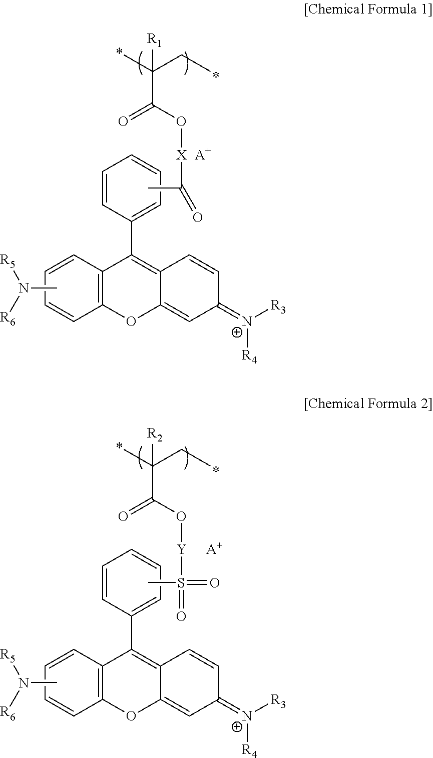

wherein,

A⁻ is selected from a halogen anion, a perhalide anion, a fluorine complex anion, an alkyl sulfate anion, a sulfonate anion and a sulfonimide anion;

R₁ and R₂ are each independently a hydrogen atom or a C₁₋₅ alkyl group;

R₃, R₄, R₅ and R₆ are each independently, a hydrogen atom, a substituted or unsubstituted C₁₋₁₈ alkyl group, a substituted or unsubstituted C₃₋₁₈ cycloalkyl group, a substituted or unsubstituted C₆₋₁₈ aryl group, a substituted or unsubstituted C₇₋₁₈ aralkyl group, a substituted or unsubstituted C₁₋₁₈ alkoxyalkyl group, or a 3 to 18-membered heterocycle substituent,

R₃ and R₄ or R₅ and R₆ form a 3 to 18-membered nitrogen-containing ring with a neighboring nitrogen atom; and

X and Y are each independently represented by the following Structural Formula 1 to Structural Formula 3:

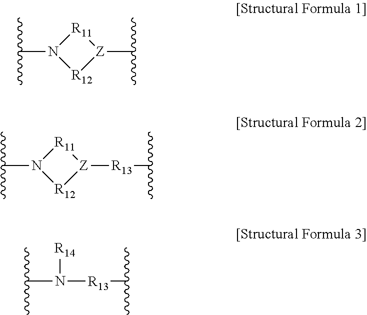

wherein,

R₁₁ and R₁₂ are each independently a substituted or unsubstituted C₁₋₁₈ alkylene group;

Z is CH or a nitrogen atom;

R₁₃ is a substituted or unsubstituted C₁₋₁₈ alkylene group, or a substituted or unsubstituted phenylene group; and

R₁₄ is a hydrogen atom, a substituted or unsubstituted C₁₋₁₈ alkyl group, a substituted or unsubstituted C₃₋₁₈ cycloalkyl group, a substituted or unsubstituted C₆₋₁₈ aryl group, a substituted or unsubstituted C₇₋₁₈ aralkyl group, a substituted or unsubstituted C₁₋₁₈ alkoxyalkyl group, or a 3 to 8-membered heterocycle substituent.

* marked in Chemical Formula represents a connecting part.

Herein, the term ‘substituted or unsubstituted’ means that at least one hydrogen atom of the corresponding substituent can be substituted with a C₁₋₆ alkyl group or an isocyanate group or not.

The polymeric compound of the present invention containing the xanthene-based compound structure represented by Chemical Formula 1 or Chemical Formula 2 may further contain a structure selected from structures of the following Chemical Formula 3 to Chemical Formula 8.

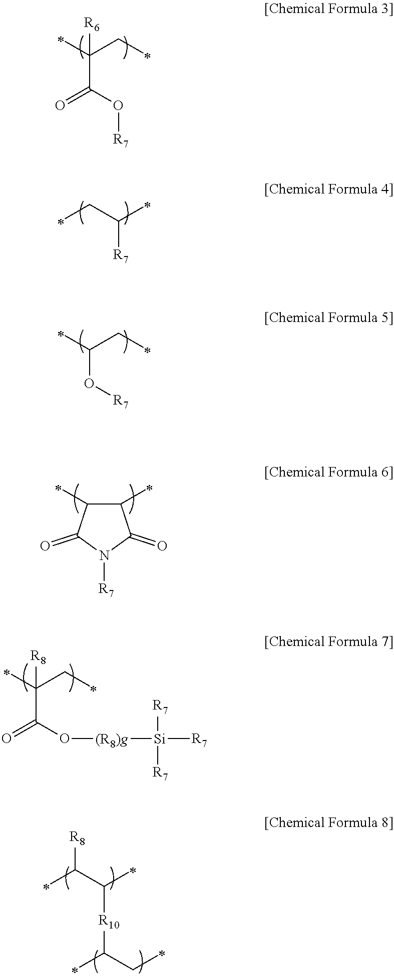

wherein,

R₇ is selected from a hydrogen atom, a substituted or unsubstituted C₁₋₁₈ alkyl group, a substituted or unsubstituted C₃₋₁₈ cycloalkyl group, a C₁₋₁₀ alkyl group substituted with a heterocycle group, a substituted or unsubstituted 3 to 8-membered heterocycle substituent, a C₃₋₁₈ cycloalkyl group substituted with an epoxy group, a substituted or unsubstituted C₆₋₁₈ aryl group, a substituted or unsubstituted C₇₋₁₈ aralkyl group, a substituted or unsubstituted C₁₋₁₈ acyl group, an allyl group and a cinnamyl group;

R₈ is a hydrogen atom or a C₁₋₅ alkyl group;

R₉ is a substituted or unsubstituted C₁₋₁₈ alkylene group;

g is 0 or 1; and

R₁₀ is a substituted or unsubstituted C₃₋₁₈ cycloalkylene group, a substituted or unsubstituted C₁₋₁₈ alkylene group or a substituted or unsubstituted C₆₋₁₈ arylene group.

Further, the A⁻ may be a fluorinated alkylsulfonic acid anion, tetrafluoroborate, tetracyanoborate, tetrakis(pentafluorophenyl)borate or an anion represented by the following Structural Formula 4:

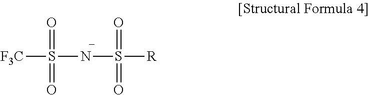

wherein,

R may be selected from a trifluoromethyl group, a substituted or unsubstituted phenyl group and a substituted or unsubstituted naphthyl.

Further, according to a preferred embodiment of the present invention, in Chemical Formula 1 and Chemical Formula 2, R₃ and R₄ may be each independently a C₁₋₁₀ alkyl group or a substituted or unsubstituted C₆₋₁₈ aryl group, or R₃ and R₄ may form a 3 to 10-membered nitrogen-containing ring with a neighboring nitrogen atom;

R₅ and R₆ may be each independently a C₁₋₁₀ alkyl group or a substituted or unsubstituted C₆₋₁₈ aryl group, or R₅ and R₆ may form a 3 to 10-membered nitrogen-containing ring with a neighboring nitrogen atom;

R₁₁ and R₁₂ may be each independently a C₁₋₆ alkylene group;

Z may be CH or a nitrogen atom;

R₁₃ may be a C₁₋₆ alkylene group; and

R₁₄ may be a hydrogen atom or a C₁₋₃ alkyl group.

Further, the compound of Chemical Formula 1 or Chemical Formula 2 is characterized that it is synthesized by a polymerization reaction using a compound represented by the following Chemical Formula 9 or Chemical Formula 10 as a monomer.

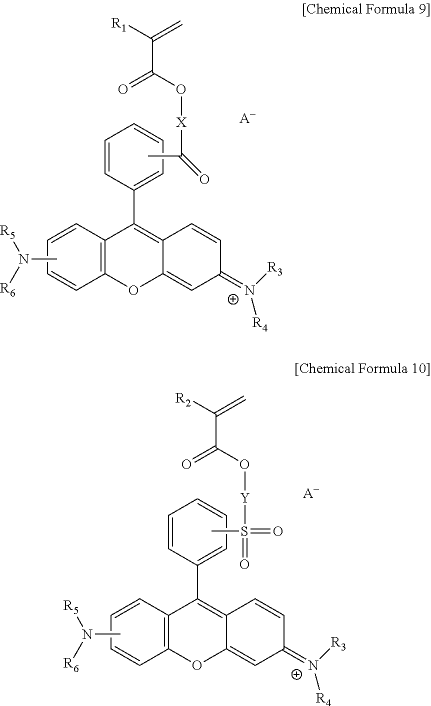

wherein,

A⁻, X, Y, R₁, R₂, R₃, R₄, R₅ and R₆ have the same meanings as defined in Chemical Formula 1 and Chemical Formula 2.

Specifically, the compound represented by Chemical Formula 9 and Chemical Formula 10 may be a compound represented by the following structure, but not limited thereto.

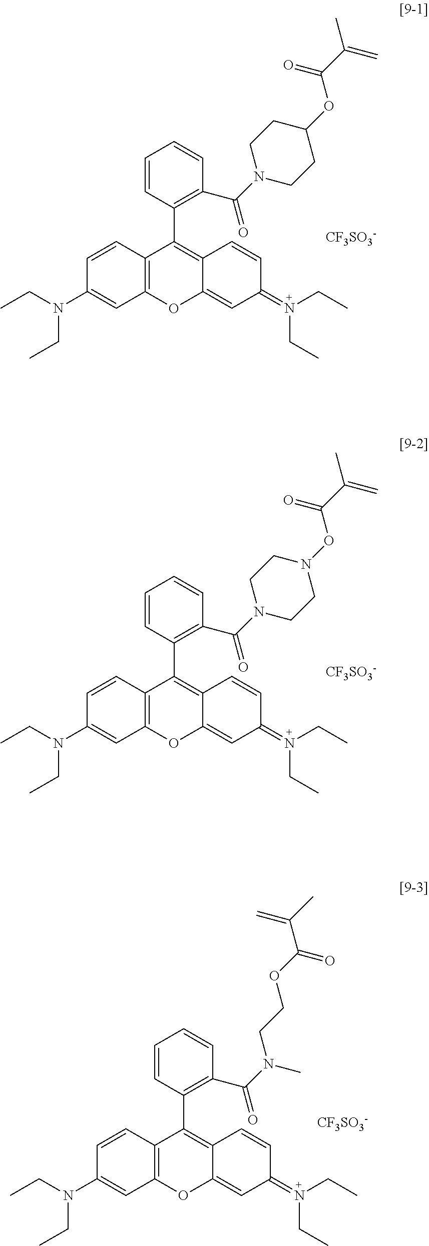

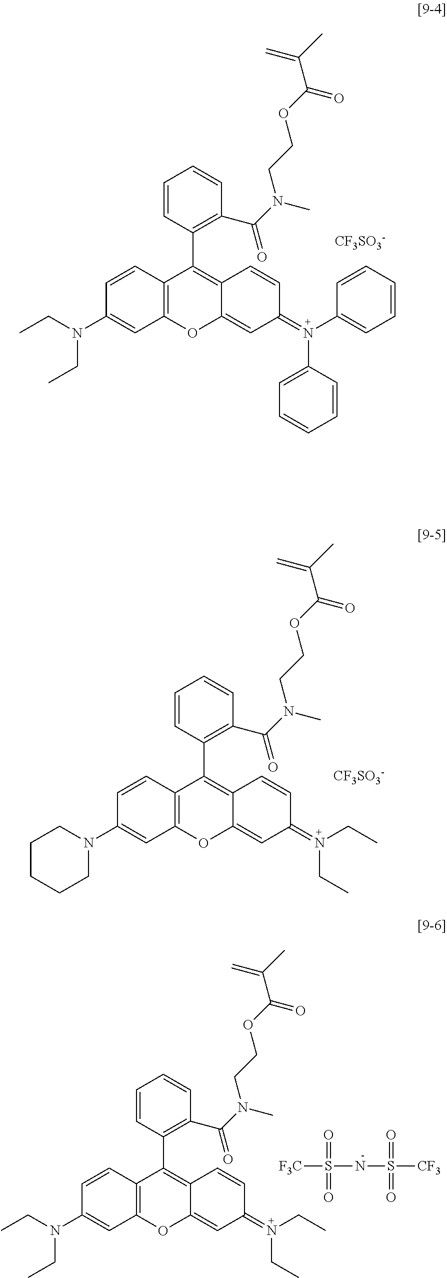

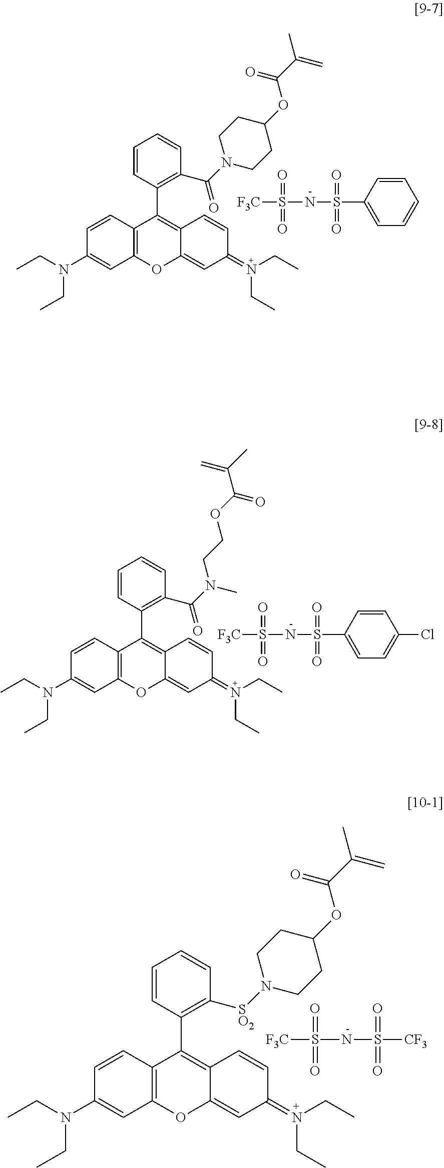

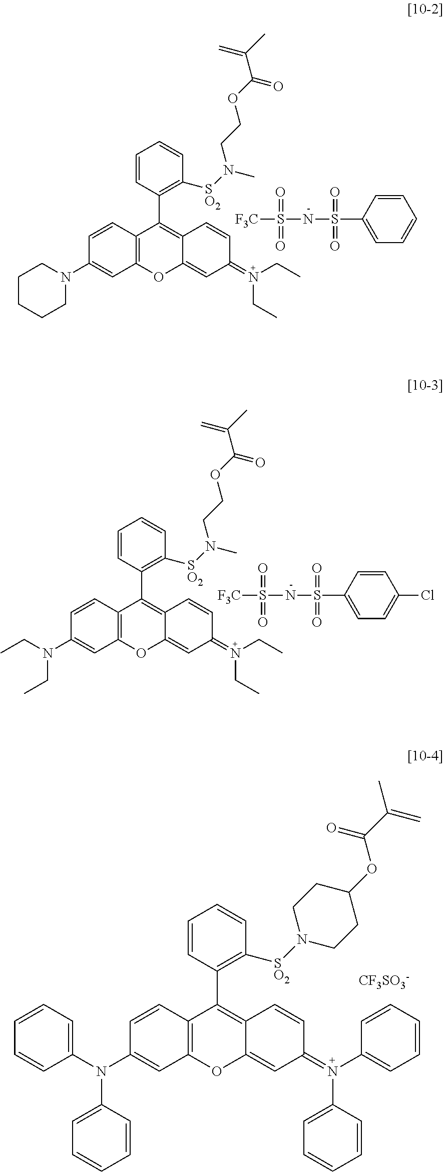

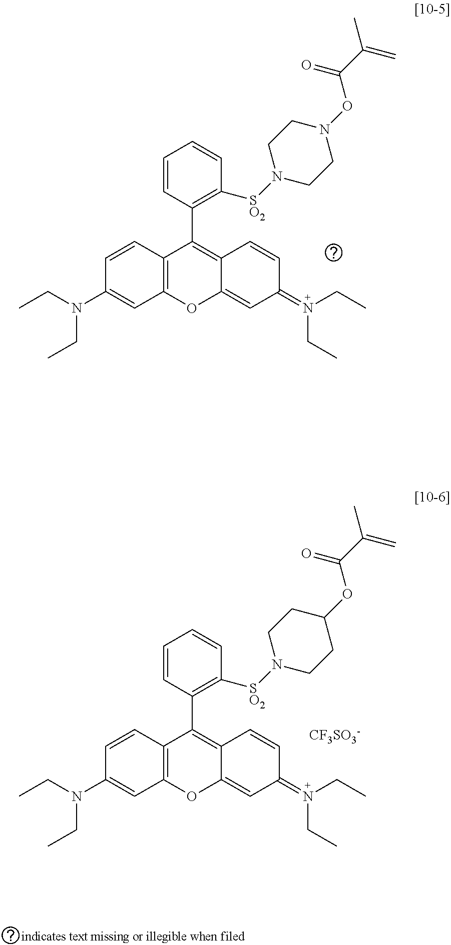

Further, the compounds of Chemical Formula 3 to Chemical Formula 8 are synthesized from monomers of the following Chemical Formula 11 to Chemical Formula 16, respectively.

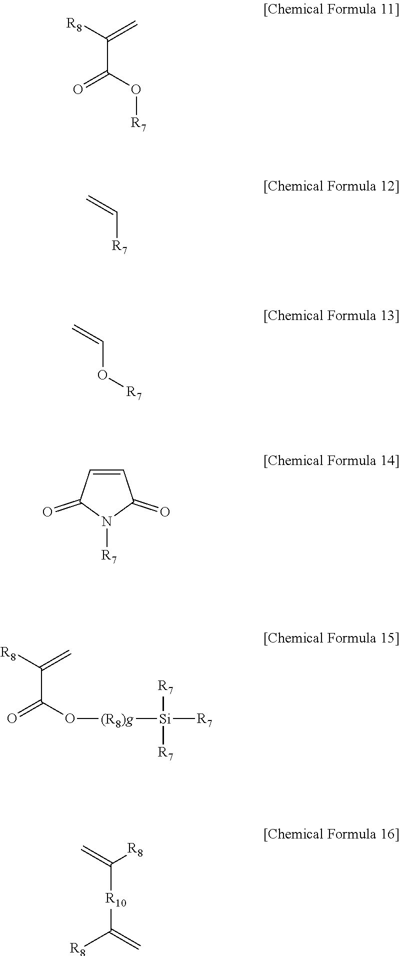

wherein,

R₇, R₈, R₉, R₁₀ and g have the same meanings as defined in Chemical Formula 3 to Chemical Formula 8.

The polymeric compound according to the present invention may have weight average molecular weight (Mw) of 2,000 to 150,000.

In another aspect, the present invention relates to a resin composition for a color filter, and the composition is characterized by comprising a xanthene-based polymeric compound, which surely contains the structure represented by Chemical Formula 1 or Chemical Formula 2, as a coloring agent. The composition may further comprise a binder resin, a reactive unsaturated compound, a polymerization initiator, an organic solvent and additives.

Further, considering color coordinate, color compensation and the like of a color filter, the coloring agent may further optionally comprise at least one kind of a dye compound or a pigment compound together with the polymeric compound of Chemical Formula 1 or Chemical Formula 2 according to the present invention.

The dye compound may be a triaryl methane dye and the like. The pigment compound may be any blue pigment without particular limitation, and preferably, it may be a compound classified as a pigment in Color Index (published by The Society of Dyers and Colourists). Specific example of the pigment compound may include Color Index (C.I.) Pigment Blue 1, 15, 15:1, 15:2, 15:3, 15:4, 15:6, 16, 60 and the like.

Further, the coloring agent may be contained in an amount of 0.01 wt% to 50 wt% based on the total weight of the resin composition for a color filter.

The binder resin is not particularly limited as long as it is a resin capable of exhibiting binding force. In particular, it may be generally known film-forming resins, and preferably may be a resin having a photopolymerizable unsaturated bond.

Specifically, the binder resin may be at least one selected from the group consisting of a cellulose resin, particularly carboxymethyl hydroxyethyl cellulose and hydroxyethyl cellulose, an acrylate resin, an alkyd resin, a melamine resin, an epoxy resin, a polyvinyl alcohol resin, a polyvinylpyrrolidone resin, a polyamide resin, a polyamide-imine resin, a polyimide resin, and the like. Preferably, it may be an acrylate resin.

More specifically, a homopolymer or a copolymer of a polymerizable monomer, e.g., a copolymer of a polymerizable monomer having a carboxyl group such as methyl methacrylate, ethyl methacrylate, propyl methacrylate, butyl methacrylate, styrene and styrene derivatives, methacrylic acid, itaconic acid, maleic acid, maleic anhydride and mono-alkyl maleate and a polymerizable monomer such as methacrylic acid, styrene and styrene derivatives may be useful. Further, the binder resin may be a reaction product of a a compound containing an oxirane ring and an ethylene-based unsaturated compound, respectively, e.g., glycidyl(meth)acrylate, acryloyl glycidyl ether, monoalkyl glycidyl itaconate and the like, and a carboxyl-containing polymeric compound, and a reaction product of a compound containing a hydroxyl group and an ethylene-based unsaturated compound (unsaturated alcohol), respectively, e.g., allyl alcohol, 2-buten-4-ol, oleyl alcohol, 2-hydroxyethyl(meth)acrylate, N-methylolacrylamide and the like, and a carboxyl-containing polymeric compound. The binder may also contain an unsaturated compound without an isocyanate group.

The unsaturation degree of the binder (the molecular weight of the binder per unsaturated compound) may be generally in a range of 200 to 3,000, particularly in a range of 230 to 1,000, to provide suitable photopolymerization properties and film hardness. The binder may have an acid value of generally 20 to 300, particularly 40 to 200, to provide sufficient alkali developing properties after exposure. The binder may have an average molecular weight of 1,500 to 200,000 g/mol, preferably 10,000 to 50,000 g/mol.

The reactive unsaturated compound may be selected from the group consisting of a thermosetting monomer or oligomer, a photocurable monomer or oligomer and a combination thereof. Preferably, it may be a photocurable monomer and may be one containing one or more reactive double bond and an additional reactive group in the molecule.

In this regard, useful photocurable monomers include, in particular, a reactive solvent or a reactive diluent, e.g., mono-, di-, tri- and poly-functional acrylate and methacrylate, vinyl ether, glycidyl ether and the like. Additional reactive groups include allyl, hydroxyl, phosphate, urethane, secondary amine, N-alkoxymethyl group and the like.

These types of monomers are known in the art, for example, see Roempp, Lexikon, Lacke and Druckfarben, Dr. Ulrich Zorll, Thimem Verlag Stuttgart-New York, 1998, pp. 491-492. Selection of the monomer depends on the type and intensity of radiation used, target reaction of a photoinitiator and desired film properties. The photocurable monomer may be used alone or in combination of the monomers.

The polymerization initiator may be a thermosetting initiator, a photocurable initiator or a combination thereof. Preferably, it may be a photocurable initiator. The photocurable initiator is a compound that forms a reaction intermediate capable of inducing polymerization of the monomer and/or the binder by absorbing visible or UV light. The photocurable initiator is also known in the art, for example, see Roempp, Lexikon, Lacke and Druckfarben, Dr. Ulrich Zorll, Thimem Verlag Stuttgart-New York, 1998, pp. 445-446.

The organic solvent may be, for example, a ketone, alkylene glycol ether, alcohol or aromatic compound. Ketones include acetone, methyl ethyl ketone, cyclohexanone and the like., alkylene glycol ethers include methyl cellosolve (ethylene glycol monomethyl ether), butyl cellosolve (ethylene glycol monobutyl ether), methyl cellosolve acetate, ethyl cellosolve acetate, butyl cellosolve acetate, ethylene glycol monopropyl ether, ethylene glycol monohexyl ether, ethylene glycol dimethyl ether, diethylene glycol ethyl ether, propylene glycol monomethyl ether, propylene glycol monoethyl ether, propylene glycol monopropyl ether, propylene glycol monobutyl ether, propylene glycol monomethyl ether acetate, diethylene glycol methyl ether acetate, diethylene glycol ethyl ether acetate, diethylene glycol propyl ether acetate, diethylene glycol isopropyl ether acetate, diethylene glycol butyl ether acetate, diethylene glycol t-butyl ether acetate, triethylene glycol methyl ether acetate, triethylene glycol ethyl ether acetate, triethylene glycol propyl ether acetate, triethylene glycol isopropyl ether acetate, triethylene glycol, triethylene glycol butyl ether acetate, triethylene glycol t-butyl ether acetate and the like. Alcohols include methyl alcohol, ethyl alcohol, isopropyl alcohol, n-butyl alcohol, 3-methyl-3-methoxybutanol and the like. Aromatic solvents include benzene, toluene, xylene, N-methyl-2-pyrrolidone, ethyl N-hydroxymethylpyrrolidone-2-acetate and the like. Examples of other solvents include 1,2-propanediol diacetate, 3-methyl-3-methyl-3-methoxybutyl acetate, ethyl acetate, tetrahydrofuran and the like. These organic solvents may be used alone or as a mixture.

The additional additives are not particularly limited so long as it does not negatively affect the desired effect. In order to improve surface texture, preferable examples include fatty acids, fatty amines, alcohols, bean oils, waxes, rosins, resins, benzotriazole derivatives and the like. More preferably, useful fatty acids may include stearic acid or behenic acid and the like, and useful fatty amines may include stearylamine and the like.

### MODE FOR INVENTION

The present invention will be explained in detail with reference to the following examples, including test examples. However, these examples are provided for illustrative purposes only and are not intended to limit the scope of the invention.

### SYNTHETIC EXAMPLE

Synthesis of dye Compound

In order to synthesize a polymeric compound, a dye compound was synthesized as the compound represented by Chemical Formula 9 and Chemical Formula 10.

### Synthetic Example 1

### Synthesis of Dye Compound 1

(1) After adding phthalic anhydride (15.7 g) and 3-(N,N-dimethylamino)phenol (15 g) to 1,2-dichlorohenzene (56.7 g) in a reactor, the resulting mixture was stirred at 175° C. for 1 hour. 1 hour later, 3-dimethylaminophenol (10 g) was added In three aliquots. After the addition was completed, the mixture was stirred at 175° C. for 12 hours. Upon completion of the reaction, the mixture was cooled to below 25° C. and, after adding 3% sodium hydroxide aqueous solution (100 g), stirred for 30 min After separating the organic layer and adding 4.5% sulfuric acid (330 g), the mixture was stirred for 30 min After separating the aqueous layer and adding 35% hydrochloric acid (30 g) and sodium chloride (15 g), the mixture was stirred at 60° C. for 1 hour. After cooling to room temperature, the resulting crystals were filtered, washed with 2% hydrochloric acid (300 g) and dried at 80° C. to obtain a compound of the following Chemical Formula A (30 g).

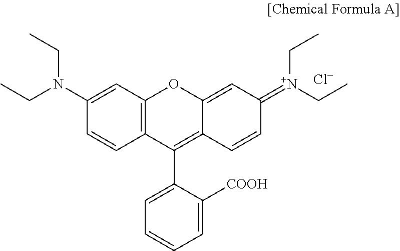 [SMILES: CCN(CC)c1ccc2c(-c3ccccc3C(=O)O)c3ccc(=[N+](CC)CC)cc-3oc2c1 ; [Cl-]]

(2) After adding the compound of Chemical Formula A obtained in 1-(1) (15.0 g) to dichloromethane (210.0 g), the mixture was stirred below 25° C. Then, thionyl chloride (12.9 g) was added dropwise below 25° C. for 30 min. After the addition was completed, N,N-dimethylformamide (1.3 g) was added dropwise below 25° C. for 30 min. After the addition was completed, reaction temperature was raised to 40° C. After stirring at the same temperature for 1 hour, the reaction mixture was cooled to below 25° C. and added to icy water (300 g). After the addition was completed, the organic layer was separated and cooled to below 0° C. Then, a mixture of 2-hydroxypiperidine and triethylamine was slowly added dropwise below 5° C. After the addition was completed, the mixture was stirred below 5° C. for 2 hours. After adding methacrylic anhydride (10 g), the mixture was heated and refluxed for 14 hours, and water (200 g) and hydrochloric acid (10 g) were added. After separating layers, the organic layer was concentrated and dried to obtain a compound: of the following Chemical Formula B (10.0 g).

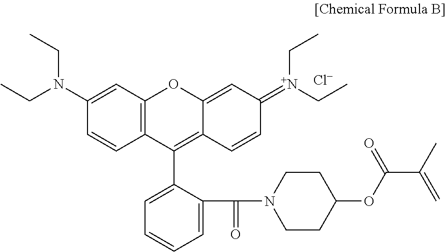 [SMILES: C=C(C)C(=O)OC1CCN(C(=O)c2ccccc2-c2c3ccc(=[N+](CC)CC)cc-3oc3cc(N(CC)CC)ccc23)CC1 ; [Cl-]]

(3) The compound of Chemical Formula B (10.0 g) obtained in 1-(2) was added to dichloromethane (100 g) and dissolved at 25° C. Lithium bis(trifluoromethane) sulfonimide (11.3 g) dissolved in water (200 g) was added dropwise to the solution of the compound of Chemical Formula B for 30 min. The mixture was stirred for about 1 hour and, upon completion of reaction, the organic layer was separated. After removing the solvent through distillation, methanol (50 g) was added and the mixture was stirred for 1 hour. The resulting mixture was added dropwise to water (200 g) for about 1 hour to precipitate crystals. The crystals were filtered, washed with water (200 g) and dried at 30° C. under reduced pressure to obtain a compound of the following dye compound 1 (9.0 g).

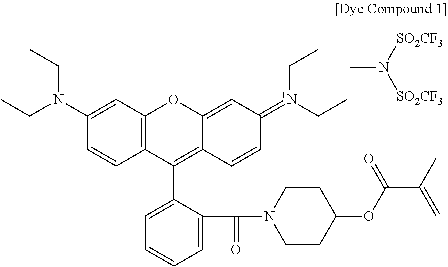

### Synthetic Example 2

### Synthesis of Dye Compounds 2 to 8

The procedure of 1-(2) to 1-(3) were repeated with the compound of Chemical Formula A obtained in 1-(1) except for using the amine and the salt listed in Table 1.

TABLE 1

| | Amine | Salt |
|---|---|---|
| Dye | 4-Hydroxypiperidine | Lithium fluorinated alkylsulfonic |
| compound 2 | | acid |
| Dye | 4-Hydroxypiperidine | Lithium tetrafluoroborate |
| compound 3 | | |
| Dye | 4-Hydroxypiperidine | Lithium tetracyanoborate |
| compound 4 | | |
| Dye | 2-Methylaminoethanol | Lithium bis(trifluoromethane) |
| compound 5 | | sulfonimide |
| Dye | 2-Methylaminoethanol | Lithium fluorinated alkylsulfonic |
| compound 6 | | acid |
| Dye | 2-Methylaminoethanol | Lithium tetrafluoroborate |
| compound 7 | | |

### Synthetic Example 3

### Synthesis of Dye Compound 8

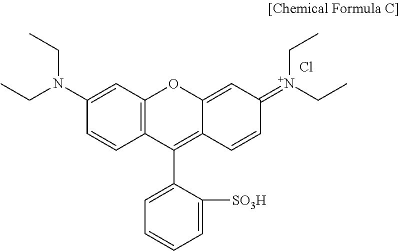 [SMILES: CCN(CC)c1ccc2c(-c3ccccc3S(=O)(=O)O)c3ccc(=[N+](CC)CC)cc-3oc2c1 ; Cl]

(1) After adding the compound of Chemical Formula C (15.0 g) to dichloromethane (210.0 g), the mixture was stirred below 25° C. Then, thionyl chloride (12.9 g) was added dropwise below 25° C. for 30 min After the addition was completed, N,N-dimethylformamide (1.3 g) was added dropwise below 25° C. for 30 min After the addition was completed, reaction temperature was raised to 40° C. After stirring at the same temperature for 1 hour, the reaction mixture was cooled to below 25° C. and added to icy water (300 g). After the addition was completed, the organic layer was separated and cooled to below 0° C. Then, a mixture of 2-hydroxypiperidine and triethylamine was slowly added dropwise below 5° C. After the addition was completed, the mixture was stirred below 5° C. for 2 hours. After adding methacrylic anhydride (10 g), the mixture was heated and refluxed for 14 hours, and water (200 g) and hydrochloric acid (10 g) were added. After separating layers, the organic layer was concentrated and dried to obtain a compound: of the following Chemical Formula D (10.0 g).

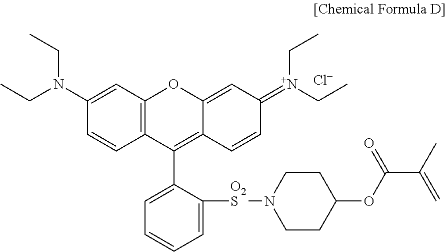 [SMILES: C=C(C)C(=O)OC1CCN(S(=O)(=O)c2ccccc2-c2c3ccc(=[N+](CC)CC)cc-3oc3cc(N(CC)CC)ccc23)CC1 ; [Cl-]]

(2) The compound of Chemical Formula D (10.0 g) obtained in 3-(1) was added to dichloromethane (100 g) and dissolved at 25° C. Lithium bis(trifluoromethane) sulfonimide (11.3 g) dissolved in water (200 g) was added dropwise to the solution of the compound of Chemical Formula D for 30 min. The mixture was stirred for about 1 hour and, upon completion of reaction, the organic layer was separated. After removing the solvent through distillation, methanol (50 g) was added and the mixture was stirred for 1 hour. The resulting mixture was added dropwise to water (200 g) for about 1 hour to precipitate crystals. The crystals were filtered, washed with water (200 g) and dried at 30° C. under reduced pressure to obtain a compound of the following dye compound 8 (8.0 g).

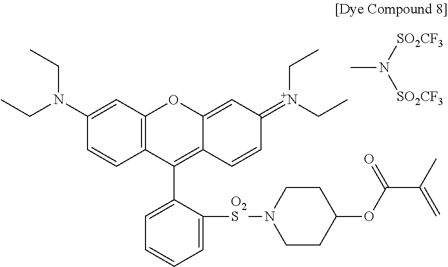

### Synthetic Example 4

### Synthesis of Dye Compounds 9 to 12

The procedure of 3-(1) to 3-(2) were repeated with the compound of Chemical Formula C except for using the amine and the salt listed in Table 2.

TABLE 2

| | Amine | Salt |
|---|---|---|
| Dye | 4-Hydroxypiperidine | Lithium fluorinated alkylsulfonic |
| compound 9 | | acid |
| Dye | 2-Methylaminoethanol | Lithium bis(trifluoromethane) |
| compound 10 | | sulfonimide |
| Dye | 2-Methylaminoethanol | Lithium fluorinated alkylsulfonic |
| compound 11 | | acid |
| Dye | 2-Methylaminoethanol | Lithium tetrafluoroborate |
| compound 12 | | |

Synthesis of Polymeric Compound

A polymeric compound was obtained by polymerizing at least one compound selected from the dye compounds 1 to 12 as the compounds represented by Chemical Formula 9 and Chemical Formula 10, or copolymerizing the compounds and at least one compound selected from the monomer compounds represented by Chemical Formula 11 to Chemical Formula 16.

At this time, the monomer compound corresponding to Chemical Formula 11 to Chemical Formula 16 were selected as shown in the following Table 3 (hereinafter, a) to q) refer to compound of Table 3).

TABLE 3

| | Compound |
|---|---|
| a) | Methyl methacrylate |
| b) | 2-Ethylhexyl acrylate |
| c) | 2-Ethylhexyl methacrylate |
| d) | Cinnamyl methacrylate |
| e) | Isobornyl methacrylate |
| f) | 1-Adamantyl methacrylate |
| g) | Benzyl methacrylate |
| h) | Glycidyl methacrylate |
| i) | 3-Methacryloxymethyl-3-ethyloxetane |
| j) | 3,4-Epoxy cyclohexylmethyl methacrylate |
| k) | Methacrylic acid |
| l) | Vinyl acetate |
| m) | Cyclohexyl vinyl ether |
| n) | Diisopropenyl benzene |
| o) | 3-(Trimethoxylsillyl)propyl methacrylate |
| p) | N-Phenyl maleimide |
| q) | 2-Isocyanate ethyl methacrylate |

### Synthetic Examples 1 to 13

According to the composition of the following Table 4, the dye compound 1 to 12 (3.0 g) and the polymerizable monomer compounds (the compounds a to q of Table 3) were put into a 50 mL flask together with AIBN (0.30 g) and MEK (44.00 g), a reflux apparatus was equipped to the flask, and then the compounds were stirred for dissolution. After nitrogen purging, the resulting solution was heated and refluxed for 24 hours to synthesize a polymeric compound. The polymeric compound thus obtained was cooled to room temperature, concentrated under reduced pressure and vacuum dried. Weight average molecular weight (Mw) and degree of dispersion of each Synthetic Example were shown in the following Table 5.

TABLE 4
(Unit: g)

| Synthetic | Dye | | | | | | | | | | | | | | | | | |
|---|---|---|---|---|---|---|---|---|---|---|---|---|---|---|---|---|---|---|
| Example | compound | a | b | c | d | e | f | g | h | i | j | k | l | m | n | o | p | r |
| 1 | 1 | 0.2 | | | | | 0.6 | | | 0.5 | | 0.7 | | | 0.8 | | | |
| 2 | 2 | | | | | | 0.4 | | | 0.5 | | 0.8 | | | 0.7 | | | |
| 3 | 3 | | | | | 0.4 | | 0.6 | | | 0.3 | 0.5 | | 0.7 | | | | |
| 4 | 4 | 0.2 | | | 0.3 | | | | 0.4 | | | 0.7 | 0.3 | | | | 0.8 | |
| 5 | 5 | | 0.9 | | | | | | | 0.5 | | 0.5 | | | | | | 0.8 |
| 6 | 6 | | | | 0.6 | | 0.6 | | | | | 0.8 | | | | 0.6 | | |
| 7 | 7 | 0.7 | | | | | | | | | | 0.5 | | 0.8 | | | 0.7 | |
| 8 | 8 | | | | 0.3 | | | 0.4 | | 1.0 | | 0.9 | | | | | | |
| 9 | 9 | | | 0.7 | | | 0.2 | | 0.9 | | | 0.7 | | | | | | |
| 10 | 10 | | | | | 0.6 | | 0.2 | | | 0.3 | 0.7 | | | 0.9 | | | |
| 11 | 11 | 0.4 | | | | | | | | | 1.0 | 0.4 | | | | 0.7 | | |
| 12 | 12 | 1.0 | | | 0.5 | | | | | | | | 0.5 | | | | 0.6 | |
| 13 | 1 | | | | | | | | | | | | | | | | | |

TABLE 5

| Synthetic | | Degree of |
|---|---|---|
| Example | Mw | Dispersion |
| 1 | 10201 | 1.85 |
| 2 | 8352 | 1.65 |
| 3 | 9468 | 1.84 |
| 4 | 7923 | 1.95 |
| 5 | 8167 | 1.67 |
| 6 | 9086 | 1.93 |
| 7 | 8795 | 1.73 |
| 8 | 7146 | 1.65 |
| 9 | 9531 | 2.16 |
| 10 | 10562 | 1.84 |
| 11 | 9956 | 1.92 |
| 12 | 8165 | 1.95 |
| 13 | 6893 | 1.66 |

Manufacture of Resin Composition for Color Filter

### Example 1

A resin composition was manufactured according to the following composition.

As a binder resin, a copolymer of benzyl methacrylate/methacrylic acid (Molecular weight ratio 60:40) (Mw=20000) (1.4 g), as an acryl monomer, dipentaerythritol hexaacrylate (5.0 g), the dye polymeric compound manufactured in Synthetic Example 1 (1.9 g), as a photopolymerization initiator, Irgacure OXE-02 (BASF) (1.0 g), and as a solvent, propyleneglycol monomethyl ether acetate (PGMEA) (40.7 g) were mixed, and then stirred for 2 hours to manufacture a resin composition.

### Examples 2 to 13

The procedure of Example 1 was repeated except for using the polymeric compound of the following Table 6 to manufacture a resin composition.

TABLE 6

| | Dye Polymeric | | Dye Polymeric |
|---|---|---|---|
| | compound | | compound |
| Example 2 | Synthetic Example 2 | Example 3 | Synthetic Example 3 |
| Example 4 | Synthetic Example 4 | Example 5 | Synthetic Example 5 |
| Example 6 | Synthetic Example 6 | Example 7 | Synthetic Example 7 |
| Example 8 | Synthetic Example 8 | Example 9 | Synthetic Example 9 |
| Example 10 | Synthetic Example | Example 11 | Synthetic Example 11 |
| | 10 | | |
| Example 12 | Synthetic Example | Example 13 | Synthetic Example 13 |
| | 12 | | |

### Comparative Example 1 to Comparative Example 4

The procedure of Example 1 was repeated with the same composition with Example 1 except for using the compound of the following Table 7 instead of the polymeric compound according to the present invention such as Synthetic Example 1 to manufacture resin compositions of Comparative Example 1 to Comparative Example 4.

TABLE 7

| | Compound | | Compound |
|---|---|---|---|
| Comparative | Rhodamine B | Comparative | Dye compound A |
| Example 1 | | Example 2 | |
| Comparative | Dye compound 4 | Comparative | Dye compound 10 |
| Example 3 | | Example 4 | |

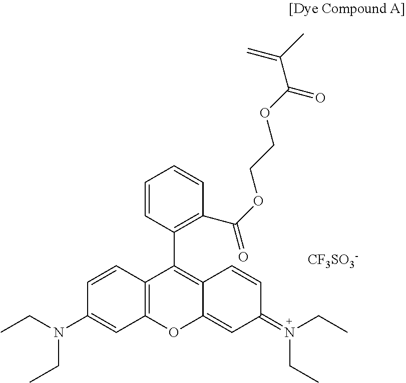

### Test Example

Measurement of Heat Resistance

For measuring heat resistance, each resin composition manufactured in Examples and Comparative Examples was spin coated on a 10 cm×10 cm glass substrate to the thickness of 2 μm, subjected to pre-bake on a 90° C. hot plate for 3 min, and then cooled at room temperature for 1 min. The substrate was exposed at a light exposure dose of 100 mJ/cm² (based on 365 nm) by using a light exposure apparatus. Then, the substrate was subjected to post-bake in a 230° C. convection oven for 30 min, and then color characteristic was checked using a spectrophotometer, MCPD3700 (Otsuka electronic). The substrate was heated in a 230° C. convection oven additionally for 1 hour, and then the color characteristic was checked again. ΔEab* value was calculated and the results were shown in the following Table 8.

Measurement of Light Resistance

The spin coating, exposing and post-baking were conducted by the same method as the method of measuring heat resistance, color characteristic was checked by using a spectrophotometer, MCPD3700 (Otsuka electronic), and then irradiated in a Q-sun Xenon Chamber at an illuminance of 1.34 W/m²/nm (at 420nm) for 8 hours, and then the color characteristic was checked again. ΔEab* value was calculated and the results were shown in Table 8.

Measurement of Chemical Resistance

The spin coating, exposing and post-baking procedures were repeated by the same method as the method of measuring heat resistance, color characteristic was checked by using a spectrophotometer, MCPD3700 (Otsuka electronic), and then the sample was immersed in propyleneglycol monomethyl ether acetate (PGMEA) at 80° C. for 30 min. Change on color characteristic before and after immersing the sample in the solvent was checked, and the results were shown in Table 8.

TABLE 8

| | Heat | Light | Chemical | | Heat | Light | Chemical |
|---|---|---|---|---|---|---|---|
| | resistance | resistance | resistance | | resistance | resistance | resistance |
| | ΔEab* | ΔEab* | ΔEab* | | ΔEab* | ΔEab* | ΔEab* |
| Example 1 | 3.51 | 4.65 | 1.28 | Example 2 | 3.38 | 3.37 | 1.53 |
| Example 3 | 3.34 | 3.08 | 1.67 | Example 4 | 3.03 | 4.59 | 1.18 |
| Example 5 | 3.26 | 4.76 | 0.65 | Example 6 | 2.01 | 3.61 | 1.84 |
| Example 7 | 1.61 | 3.34 | 2.48 | Example 8 | 1.68 | 3.28 | 0.89 |
| Example 9 | 2.68 | 4.37 | 0.73 | Example 10 | 1.39 | 3.67 | 1.95 |
| Example 11 | 2.75 | 3.67 | 1.61 | Example 12 | 1.34 | 3.63 | 2.67 |
| Example 13 | 4.36 | 4.62 | 6.07 | Comp. | 46.85 | 17.56 | 75.85 |
| | | | | Example 1 | | | |
| Comp. | 2.64 | 3.05 | 58.28 | Comp. | 1.93 | 3.51 | 41.58 |
| Example 2 | | | | Example 3 | | | |
| Comp. | 2.53 | 4.84 | 47.25 | | | | |
| Example 4 | | | | | | | |

As shown in the Table 8, it can be found that the samples of Example 1 to Example 13 according to the present invention had very excellent heat resistance, light resistance and chemical resistance, compared to the sample of Comparative Example 1. Further, it can be found that the samples of Comparative Examples 2 to 4 had good heat resistance and light resistance, but very poor chemical resistance.

Although specific embodiments of the present invention are described in detail as described above, it will be apparent to those skilled in the art that the specific description is merely desirable exemplary embodiment and should not be construed as limiting the scope of the present invention. Therefore, the substantial scope of the present invention is defined by the accompanying claims and equivalent thereof.

1. A polymeric compound comprising a structure represented by the following Chemical Formula 1 or Chemical Formula 2: 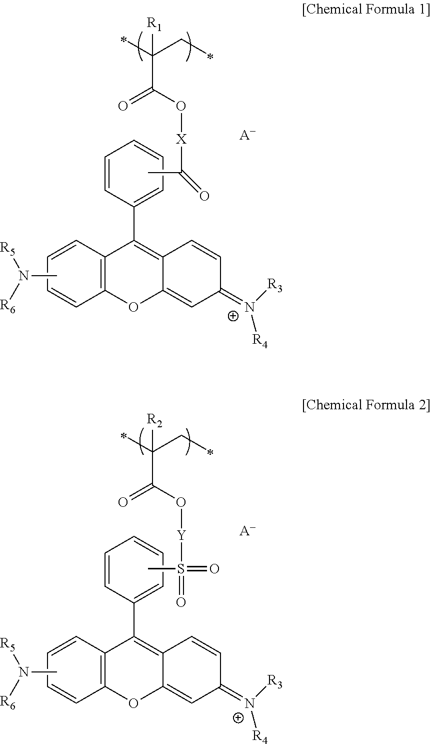
wherein,
R₁ and R₂ are each independently a hydrogen atom or a C₁₋₅ alkyl group;
A⁻ is selected from a halogen anion, a perhalide anion, a fluorine complex anion, an alkyl sulfate anion, a sulfonate anion and a sulfonimide anion;
R₃, R₄, Rs and R₆ are each independently, a hydrogen atom, a substituted or unsubstituted C₁₋₁₈ alkyl group, a substituted or unsubstituted C₃₋₁₈ cycloalkyl group, a substituted or unsubstituted C₆₋₁₈ aryl group, a substituted or unsubstituted C₇₋₁₈ aralkyl group, a substituted or unsubstituted C₁₋₁₈ alkoxyalkyl group, or a 3 to 18-membered heterocycle substituent,
R₃ and R₄ or R₅ and R₆ form a 3 to 18-membered nitrogen-containing ring with a neighboring nitrogen atom; and
X and Y are each independently represented by the following Structural Formula 1 to Structural Formula 3: 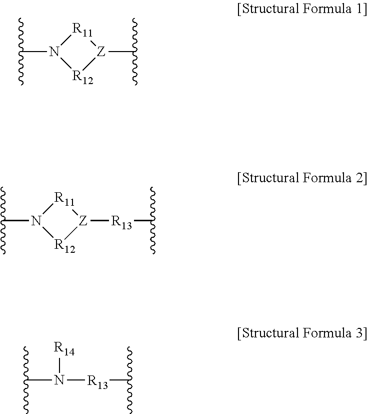
wherein,
R₁₁ and R₁₂ are each independently a substituted or unsubstituted C₁₋₁₈ alkylene group;
Z is CH or a nitrogen atom;
R₁₃ is a substituted or unsubstituted C₁₋₁₈ alkylene group, or a substituted or unsubstituted phenylene group; and
R₁₄ is a hydrogen atom, a substituted or unsubstituted C₁₋₁₈ alkyl group, a substituted or unsubstituted C₃₋₁₈ cycloalkyl group, a substituted or unsubstituted C₆₋₁₈ aryl group, a substituted or unsubstituted C₇₋₁₈ aralkyl group, a substituted or unsubstituted C₁₋₁₈ alkoxyalkyl group, or a 3 to 8-membered heterocycle substituent.

2. The polymeric compound according to claim 1, which further comprises at least one structure selected from structures of the following Chemical Formula 3 to Chemical Formula 8: 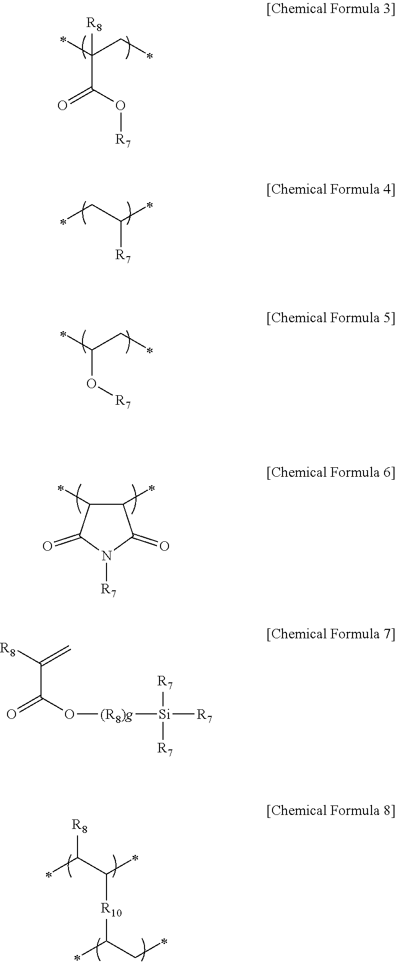
wherein,
R₇ is selected from a hydrogen atom, a substituted or unsubstituted C₁₋₁₈ alkyl group, a substituted or unsubstituted C₃₋₁₈ cycloalkyl group, a C₁₋₁₀ alkyl group substituted with a heterocycle group, a substituted or unsubstituted 3 to 8-membered heterocycle substituent, a C₃₋₁₈ cycloalkyl group substituted with an epoxy group, a substituted or unsubstituted C₆₋₁₈ aryl group, a substituted or unsubstituted C₇₋₁₈ aralkyl group, a substituted or unsubstituted C₁₋₁₈ acyl group, an allyl group and a cinnamyl group;
R₈ is a hydrogen atom or a C₁₋₅ alkyl group;
R₉ is a substituted or unsubstituted C₁₋₁₈ alkylene group;
g is 0 or 1; and
R₁₀ is a substituted or unsubstituted C₃₋₁₈ cycloalkylene group, a substituted or unsubstituted C₁₋₁₈ alkylene group or a substituted or unsubstituted C₆₋₁₈ arylene group.

3. The polymeric compound according to claim 1, wherein A⁻ is a fluorinated alkylsulfonic acid anion, tetrafluoroborate, tetracyanoborate, tetrakis(pentafluorophenyl)borate or an anion represented by the following Structural Formula 4: 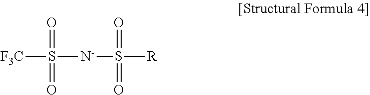
wherein,
R is selected from a trifluoromethyl group, a substituted or unsubstituted phenyl group and a substituted or unsubstituted naphthyl.

4. The polymeric compound according to claim 1, wherein R₃ and R₄ are each independently a C₁₋₁₀ alkyl group or a substituted or unsubstituted C₆₋₁₈ aryl group, or R₃ and R₄ form a 3 to 10-membered nitrogen-containing ring with a neighboring nitrogen atom;
R₅ and R₆ are each independently a C₁₋₁₀ alkyl group or a substituted or unsubstituted C₆₋₁₈ aryl group, or R₅ and R₆ form a 3 to 10-membered nitrogen-containing ring with a neighboring nitrogen atom;
R₁₁ and R₁₂ are each independently a C₁₋₆ alkylene group;
Z is CH or a nitrogen atom;
R₁₃ is a C₁₋₆ alkylene group; and
R₁₄ is a hydrogen atom or a C₁₋₃ alkyl group.

5. The polymeric compound according to claim 1, wherein the compounds of Chemical Formula 1 or Chemical Formula 2 are synthesized from a monomer represented by the following Chemical Formula 9 or Chemical Formula 10: 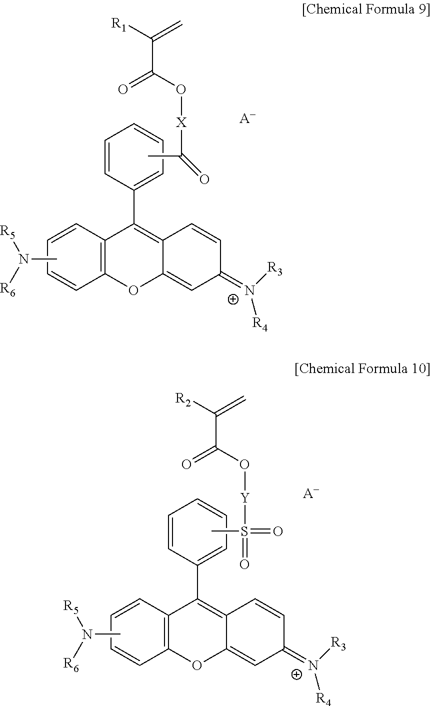
wherein,
A⁻, X, Y, R₁, R₂, R₃, R₄, R₅ and R₆ have the same meanings as defined in Chemical Formula 1 and Chemical Formula 2.

6. The polymeric compound according to claim 2, wherein the compounds of Chemical Formula 3 to Chemical Formula 8 are synthesized from a monomer represented by the following Chemical Formula 11 to Chemical Formula 16, respectively: 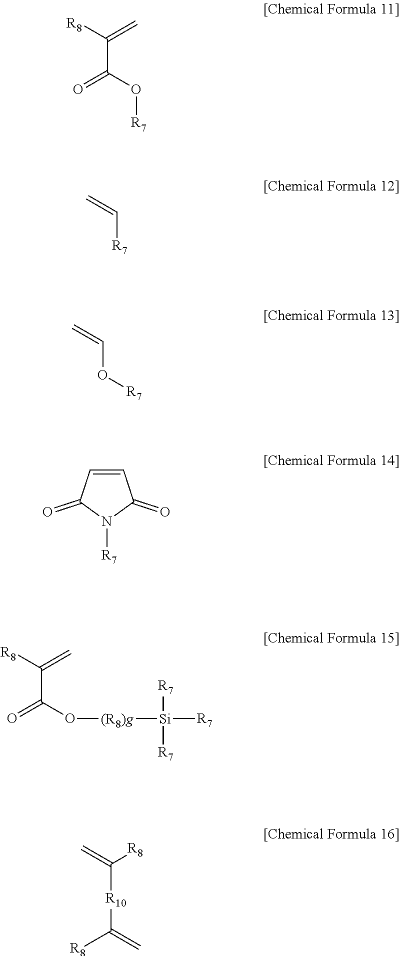
wherein,
R₇, R₈, R₉, R₁₀ and g have the same meanings as defined in Chemical Formula 3 to Chemical Formula 8.

7. The polymeric compound according to claim 1, wherein the polymeric compound has weight average molecular weight (Mw) of 2,000 to 150,000.

8. A resin composition for a color filter comprising a coloring agent, a binder resin, a reactive unsaturated compound, a polymerization initiator, an organic solvent and additives,
wherein the coloring agent comprises the polymeric compound according to claim 1.

9. The resin composition for a color filter according to claim 8, wherein the coloring agent further comprises a pigment compound, a dye compound or a mixture thereof.

10. The resin composition for a color filter according to claim 8, wherein the coloring agent is contained in an amount of 0.01 wt % to 50 wt % based on the total weight of the resin composition.

11. The resin composition for a color filter according to claim 8, wherein the reactive unsaturated compound is at least one selected from the group consisting of a thermosetting monomer or oligomer, a photocurable monomer or oligomer and a combination thereof.

12. The resin composition for a color filter according to claim 8, wherein the polymerization initiator is at least one selected from the group consisting of a thermal polymerization initiator, a photopolymerization initiator and a combination thereof.

13. A color filter prepared by using the dye polymeric compound according to claim 1 as a coloring agent.

14. A xanthene-based compound represented by the following Chemical Formula 9 or Chemical Formula 10: 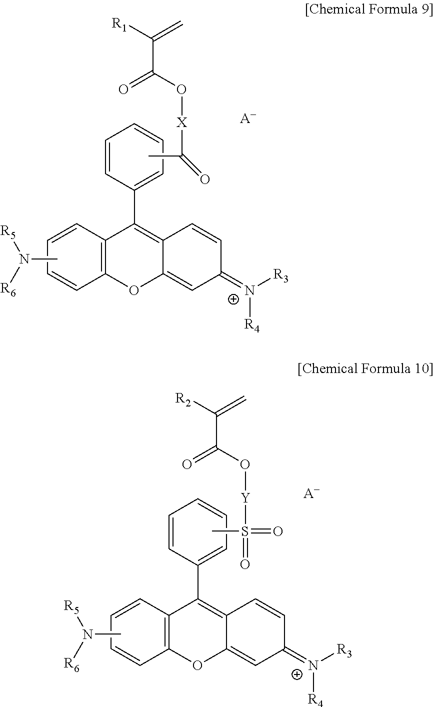
wherein,
A⁻, X, Y, R₁, R₂, R₃, R₄ and R₆ have the same meanings as defined in Chemical Formula 1 and Chemical Formula 2.
